(19) United States
(12) Patent Application Publication
(10) Pub. No.: US 2003/0092939 A1
(43) Pub. Date: May 15, 2003

(54) Preparation of metal formate/formic acid mixtures

(76) Inventors: Eckhard Strofer, Mannheim (DE); Karl-Heinz Wostbrock, Morstadt (DE); Markus Weber, Ludwigshafen (DE)

Correspondence Address: KEIL & WEINKAUF, 1350 CONNECTICUT AVENUE, N.W., WASHINGTON, DC, 20036, US

(21) Appl. No.: 10/289,111

(22) Filed: Nov. 7, 2002

(30) Foreign Application Priority Data
Nov. 9, 2001 (DE) 10154757.9

(51) Int. Cl. C07C 53/06

(52) U.S. Cl. 562/609

(57) ABSTRACT

A process is described for preparing metal formate/formic acid mixtures comprising the following steps:

a) Reacting at least one metal hydroxide with carbon monoxide or a carbon monoxide-containing gas in the presence of a catalyst in an aqueous solution to give the corresponding metal formate(s);

b) separating off the water and catalyst by distillation;

c) adding formic acid;

d) if appropriate, adjusting to storage temperature the temperature of the metal formate/formic acid mixture obtained using steps a) to c);

with, in step a), the metal hydroxides being the hydroxides of alkali metals and/or alkaline earth metals and step c) also being able to be carried out in time before step b).

The catalyst is at least one catalyst selected from the group consisting of alcohol and formic esters. Preferred catalysts are unbranched C₁-C₄ alkanols, branched C₃-C₄ alkanols and their formic esters. Preferred metal hydroxides are the hydroxides of sodium, potassium and/or calcium. The concentration of the formic acid used in step c) is generally from 80 to 99.99% by weight in water, and the storage temperature in step d) is a temperature from 0 to 40° C.

The invention relates to a process for preparing metal formate/formic acid mixtures, the metal formate being obtained by reacting metal hydroxides with carbon monoxide (CO) in the presence of a catalyst. Starting materials of the hydroxides of alkali metals and/or alkaline earth metals, in particular of sodium (Na), potassium (K) and/or calcium (Ca).

Metal formate/formic acid mixtures are of great importance, for example, as fertilizers and animal feed additives. When used as animal feed additives, attention must be paid to the fact that the metal formates can react with formic acid, forming disalts of formic acid, so that no formic acid is then released from the mixture. Potassium formate itself is also used as an oil field chemical (drilling fluid).

In the prior art, processes for preparing metal formates from metal hydroxides and carbon monoxides are already known.

According to A. F. Hollemann, N. Wiberg, Lehrbuch der anorganischen Chemie [Textbook of Inorganic Chemistry], Walter de Gruyter Verlag Berlin New York, 1985, 91st-100th edition, page 722, sodium formate may be prepared by introducing carbon monoxide into sodium hydroxide solution at from 150 to 170° C. and a pressure of from 3 to 4 bar. According to the same textbook, page 947, potassium formate is produced by the action of carbon monoxide on an aqueous solution of potassium sulfate and quick lime at 230° C. and 30 bar.

In the process of U.S. Pat. No. 4,157,246 for coke processing, the formation of water-soluble alkali metal formates is used for recovering the alkali metals such as potassium and cesium from the alkali-metal-containing catalysts used. For this the alkali-metal-containing catalyst residues are treated with water and calcium compounds and/or magnesium compounds such as calcium hydroxide or magnesium hydroxide; carbon monoxide is introduced, with or without the addition of alkali metal sulfate, and this mixture is reacted at from approximately 140 to 390° C., forming water-soluble alkali metal formates. When alkali metal sulfates are added, these react with calcium hydroxide to form calcium sulfate. Since calcium sulfate precipitates out of the reaction solution, this shifts the reaction equilibrium toward the product side.

U.S. Pat. No. 3,262,973 describes a process for preparing alkali metal formates and ammonium formates starting from alkali metal hydroxide and ammonium hydroxide and also carbon monoxide in an alcoholic solution which may contain water. The alcohols used are, for example, C₁-C₄ alkanols, cyclohexanol, furfuryl alcohol and benzyl alcohol. Either pure carbon monoxide or a gas mixture containing carbon monoxide can be used, so that the process can also be used for removing CO from gas mixtures. Since the alkali metal formates and ammonium formate, in contrast to the alkali metal hydroxides and ammonium hydroxide, are insoluble in the alcoholic solution, an alkali metal formate or ammonium formate precipitate forms which, by decanting, filtering or centrifuging, can be separated off from the mother liquor which can be recycled. The process can be carried out batchwise and continuously. The reaction temperatures are variable within broad ranges. Generally, at atmospheric pressure, they are from 0° C. to the boiling point of the alcohol used. Preference is given to temperatures of from 10 to 30° C. In the examples, the reaction of from 8 to 10% strength by weight ethanolic NaOH or KOH solution is described.

Processes for preparing formate/formic acid mixtures are also already known in the literature. According to the process of WO 96/35657, potassium hydroxide, carbonate, hydrogen carbonate or formate, sodium hydroxide, carbonate, hydrogen carbonate or formate, cesium hydroxide, carbonate, hydrogen carbonate or formate, ammonium formate or ammonia are mixed together with formic acid containing from 0 to 50% water at from 40 to 100° C., cooled, filtered and dried, and in this manner, ultimately, the corresponding disalts of formic acid are obtained. However, since in the case of the metal hydroxides, carbonates or hydrogen carbonates, in each case 1 mol of formic acid is used for producing the appropriate formate from the metal salt, this process is relatively expensive.

It is an object of the present invention to provide a process by which metal formate/formic acid mixtures can be prepared inexpensively on an industrial scale, with the metal formates being the formates of alkali metals and/or alkaline earth metals, in particular the formates of Na, K and/or Ca.

We have found that this object is achieved according to the invention by a process for preparing metal formate/formic acid mixtures comprising the following steps:

a) Reacting at least one metal hydroxide with carbon monoxide (CO) or a CO-containing gas in the presence of a catalyst in an aqueous solution to give the corresponding metal formate(s);

b) separating off the water and catalyst by distillation;

c) adding formic acid;

d) if appropriate, adjusting to storage temperature the temperature of the metal formate/formic acid mixture obtained using steps a) to c);

with, in step a), the metal hydroxides being the hydroxides of alkali metals and/or alkaline earth metals and step c) also being able to be carried out in time before step b).

The inventive process permits the inexpensive preparation of metal formate/formic acid mixtures on an industrial scale.

Process steps a) to d) will now be described individually in more detail.

Step a)

The hydroxides of alkali metals and/or of alkaline earth metals are generally reacted with CO or a CO-containing gas in the presence of a catalyst in aqueous solution to give the corresponding metal formate(s) at temperatures of from 20 to 250° C., preferably from 30 to 160° C., particularly preferably from 90 to 120° C., and pressures of from 1 to 50 bar, preferably from 3 to 40 bar, particularly preferably from 8 to 20 bar.

Compared with otherwise customary processes for formate preparation, higher concentration ranges of the metal hydroxide can be employed, and there is a trend toward higher pressures and lower temperatures being able to be employed. Since the reaction is limited by mass transfer, higher space-time yields may be achieved by very thorough mixing, for example using mixing nozzles.

The reaction can be carried out both continuously and batchwise. Preference is given to the continuous reaction. Generally, the reaction is carried out in such a manner that the metal hydroxide is virtually quantitatively converted to the metal formate. The end of the reaction may be established by determining the proportion of metal hydroxide in the solution, for example by titration. The reaction is advantageously carried out until the proportion of metal hydroxide in the reaction solution is less than 0.1% by weight, preferably less than 0.04% by weight, particularly preferably less than 0.01% by weight.

The reaction can in principle be carried out in any type of reaction vessel. Preferably, it is carried out in a stirred tank having a gas-introduction device, in a bubble column or in a loop reactor. Particularly preferably, the reaction is carried out in a loop reactor or a bubble column, very particularly preferably in a loop reactor, since here, owing to the high interfacial area between the aqueous solution containing the metal hydroxide and catalyst and the CO introduced or the CO-containing gas, a high absorption rate and thus also a high reaction rate result.

The metal hydroxides are generally used in aqueous solution. The concentration of these metal hydroxide solutions is generally from 25 to 50% by weight, preferably from 45 to 50% by weight, particularly preferably from 48.5 to 50% by weight. The aqueous solution can also contain a plurality of metal hydroxides. Generally, no special requirements are made as to the purity of the metal hydroxide solutions used. Generally, therefore, technical grade metal hydroxide solutions are used. These can be contaminated, for example, with a content of sodium ions and carbonate ions <0.1% by weight, or a content of chloride ions and sulfate ions <25 mg/kg of metal hydroxide. The inventive process may also be carried out using pure metal hydroxide solutions. Preference is given to hydroxides of sodium, potassium and/or calcium, particular preference is given to potassium hydroxide.

CO can be used not only as an individual component, but also in a mixture with other gases. Such other gases are gases which are inert under the reaction conditions, for example N₂ and the noble gases.

If CO is used in a mixture with other gases, the proportion of CO in the gas mixture is at least 5% by volume, preferably at least 10% by volume, particularly preferably at least 25% by volume, very particularly preferably at least 50% by volume. The CO partial pressure during the reaction is generally from 1 to 60 bar, preferably from 20 to 55 bar, particularly preferably from 40 to 55 bar.

Generally, no special requirements are made of the purity of the CO used or of a corresponding CO-containing gas mixture. The reaction may hence be carried out not only with pure CO but also with technical grade CO or the corresponding CO-containing gas mixtures. Preferably, pure CO or a CO-containing gas mixture having a CO content of at least 50% by volume is used, particularly preferably pure CO is used.

CO or the CO-containing gas mixture can be fed into the reactor not only from the top but also from below, that is to say in cocurrent or countercurrent flow. It is also possible to inject CO directly into the metal hydroxide line or catalyst line and to feed CO intermediately, that is to say via a side stream feed.

The catalyst is at least one catalyst selected from the group consisting of alcohol and formic acid esters. In principle, all catalysts are suitable in which the metal hydroxides dissolve well. Suitable catalysts are, for example, saturated unbranched C₁-C₄ alkanols, unsaturated unbranched C₃-C₄ alkanols, saturated branched C₃-C₄ alkanols and unsaturated branched C₄ alkanols and formic esters thereof. If an alcohol is used as catalyst in a mixture with a formic ester, generally the formic ester of this alcohol is used. Preference is given to saturated unbranched C₁-C₄ alkanols and saturated branched C₃-C₄ alkanols, particularly preferably, methanol is used. The catalyst is generally used as a concentration of from 2 to 40% by weight, preferably from 10 to 25% by weight, particularly preferably from 15 to 20% by weight, based on the total reaction solution.

Also, no special requirements are made of the purity of the catalyst, as is the case with the metal hydroxide and the CO or CO-containing gas mixture. Generally, therefore, technical grade alcohols and/or their formic esters are used.

The metal formates obtained according to step a) are generally present in the reaction solution at a concentration of from 10 to 90% by weight, preferably from 30 to 80% by weight, particularly preferably from 40 to 70% by weight.

The reaction conditions ensure that metal formate does not crystallize out or precipitate out, so that the reaction mixture is a solution for the duration of the reaction. Precipitated substances and line blockages caused by them can otherwise lead to expensive and time-consuming production outages. This is avoided in the inventive process.

Step b)

To recover the metal formate, water and catalyst are removed from the reaction solution. Since water and the catalyst have the lowest boiling points in the reaction solution obtained by step a), they may be simply separated off by distillation, in which case the distillation can be performed in any design of distillation column into which the reaction mixture is transferred after completion of step a). Also, more than one distillation column can be used. In this case the distillation columns are connected to one another.

Water and catalyst may be separated off not only simultaneously, under some circumstances as an azeotrope, but also successively.

In one embodiment, the catalyst and the water are taken off together overhead from a first distillation column and further fractionated in a further column. The metal formate melt is taken off via the bottom of the first distillation column.

In another embodiment, the catalyst is taken off overhead from the distillation column and water and metal formate are taken off via the bottom of the distillation column and separated from one another in a second column.

In a third variant, a dividing wall column is used. Here, catalyst, metal formate and water can be separated from one another in a single step. The use of a dividing wall column is preferred, since it is particularly cost-effective.

In all three variants, the metal formate is produced in the bottom of a distillation column as metal formate melt. The water content of the metal formate melt is generally less than 2% by weight, preferably less than 0.5% by weight, particularly preferably less than 0.1% by weight.

The catalyst is preferably recycled to the reactor in whole or in part, possibly as aqueous solution. The water can be reused in whole or in part to produce a solution of the metal hydroxide, or can be supplied to other treatment devices.

The distillation is generally carried out at a pressure of from 0.2 to 15 bar, preferably from 1 to 5 bar, particularly preferably from 1 to 3 bar. The distillation temperature is generally from 50 to 250° C., preferably from 60 to 150° C., particularly preferably from 90 to 120° C.

Step c)

The metal formate melt produced in the bottom of the distillation column, which melt, at atmospheric pressure, generally has a temperature from 100 to 255° C., preferably a temperature from 120 to 180° C., particularly preferably from 140 to 160° C., is then transferred to any design of mixer, if appropriate brought to a temperature from 110 to 120° C., preferably to a temperature from 115° C. to 120° C., and, in the mixer, mixed with formic acid at temperatures from 8° C. to 120° C., preferably at temperatures from 50° C. to 120° C., particularly preferably at temperatures from 90° C. to 120° C., very particularly preferably at temperatures from 100° C. to 120° C. The upper temperature limit is given by the decomposition temperature of formic acid of 120° C., and the lower limit by the melting point of formic acid of 8° C. The pressure during the mixing operation is generally from 0.5 to 10 bar, preferably from 1 to 5 bar, particularly preferably from 1 to 2 bar.

The formic acid used is generally an aqueous solution having a concentration from 80 to 99.9% by weight, preferably from 94 to 99.9% by weight, particularly preferably from 98 to 99.99% by weight. Technical grade purity is sufficient; pure (aqueous) formic acid may also be used, however.

Since formic acid is corrosive, the mixer should consist of a formic-acid-resistant material, for example of austenitic chrome-nickel steel or highly alloyed stainless steels. Highly alloyed stainless steels are preferred.

The mixer used may be any desired type of stirred tank, extruder or static mixer, with or without heat exchange devices, in which case mixing is performed using reaction mixing pumps, mixing nozzles or fluidized beds. Preferably static mixers, with or without heat exchange devices, are used.

When potassium formate and/or sodium formate are used, the mixing operation can also be performed according to the methods described in WO 96/35657.

Mixing the metal formates with formic acid produces disalts of the formic acid.

In the inventive process, step c) may also be carried out before step b) in time. However, the time sequence a), b), c) is preferred.

The formic acid concentration, the temperature and the pressure during the mixing operation are independent of whether step c) is carried out before or after step b).

When the inventive process is carried out in the sequence of steps a), c), b), the reaction solution taken off from the reactor after carrying out step a) is fed to a mixer, if appropriate at temperatures from 110 to 120° C., preferably at temperatures from 115 to 120° C., and mixed with formic acid in one of the abovedescribed mixers. The temperature during the mixing operation is generally from 8 to 120° C., preferably from 50 to 120° C., particularly preferably from 90 to 120° C., very particularly preferably from 100 to 120° C. The pressure is from 0.5 to 10 bar, preferably from 1 to 5 bar, particularly preferably from 1 to 2 bar.

This reaction mixture is then fed to a separation unit in which water and catalyst and also low-boiling impurities such as formaldehyde which are possibly present in the formic acid are distilled off. The catalyst is preferably recycled in whole or in part to the reactor as aqueous solution. The water may be reused in whole or in part for producing metal hydroxide solutions. The impurities of the formic acid which are separated off are discarded.

Since formic acid is then present in this reaction mixture, it must be ensured that the separation unit also comprises one of the abovementioned formic-acid-resistant materials. A suitable separation unit is any type of distillation column. However, more than one distillation column can be used. In this case, the distillation columns are connected to one another. Preferably, a dividing wall column is used, which is particularly cost-effective.

Step d)

The temperature of the metal formate/formic acid mixtures obtained by steps a) to c) is adjusted to storage temperature. Storage temperature, for the purposes of the present invention is a temperature from 0 to 40° C., preferably from 20 to 30° C., particularly preferably from 20 to 25° C. Suitable heat exchangers are all customary commercially available heat exchangers; for example, a fluidized bed is used.

The metal formate/formic acid mixtures brought to storage temperature in this manner are then supplied to the store or dispatched in solid form, that is to say, for example, as crystals or granules.

Drying may be performed in this case according to the procedure described in WO 96/35657, in which case a desiccant can also be added to the metal formate/formic acid mixture. From time to time anticaking agent is also added to the mixture.

The residual water content in the metal formate/formic acid mixture is generally less than 0.5% by weight.

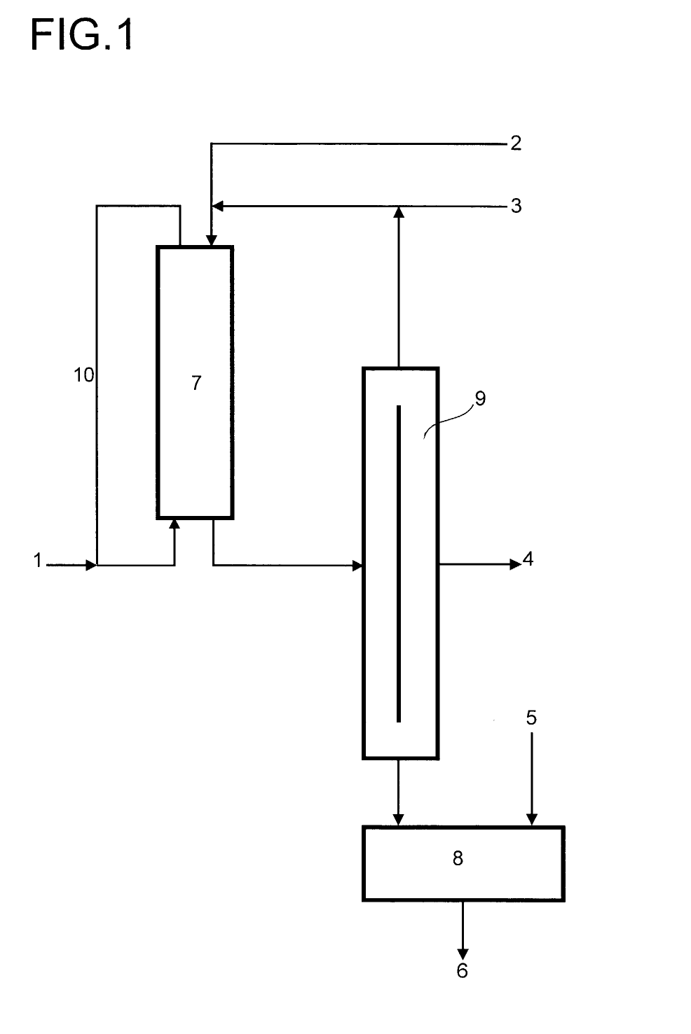

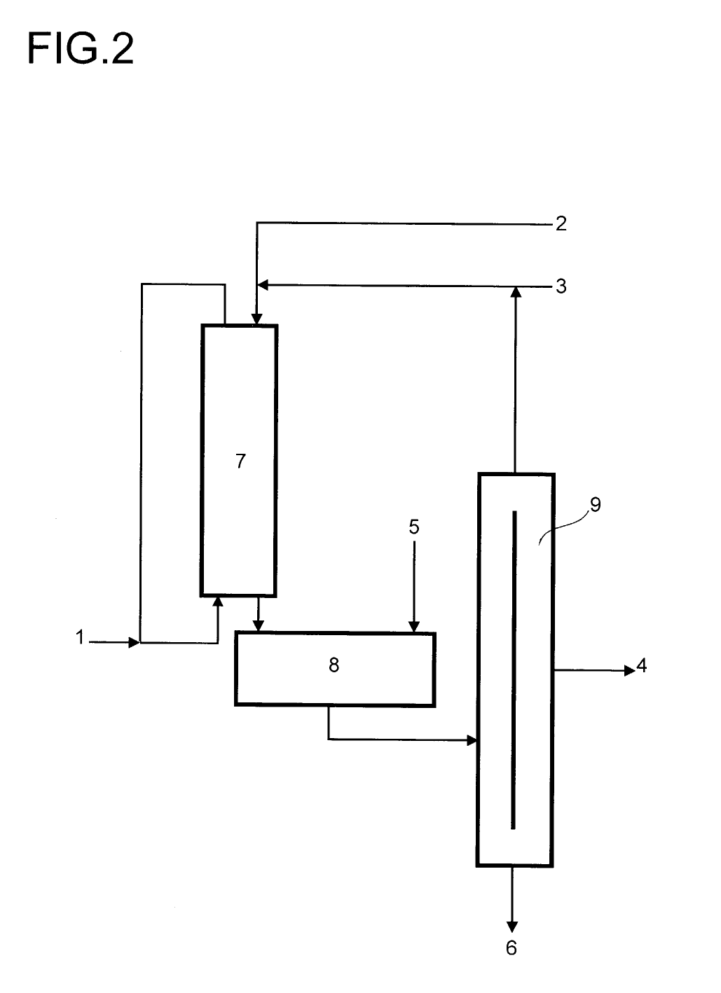

The accompanying drawing shows, in FIGS. 1 and 2, diagrammatic plants in which the inventive process is preferably carried out, that is to say,

FIG. 1 shows a diagrammatic representation of a plant for producing metal formate/formic acid mixtures for the continuous process, in which step c) is carried out after step b).

FIG. 2 shows a diagrammatic representation of a plant for producing metal formate/formic acid mixtures for the continuous process, in which step c) is carried out before step b).

In a plant according to FIG. 1, the individual process steps are carried out in the time sequence a), b) and c). CO or a CO-containing gas mixture is passed into a reactor 7 via an inlet 1 from the bottom in countercurrent flow and unreacted CO is taken off from the reactor from the top and in whole or in part recycled to the reactor 7 via a line 10. A catalyst stream is added via a feedline 3 to an aqueous metal hydroxide solution in a feedline 2 and passed into the reactor 7 from the top. The reaction is generally carried out at a temperature from 20 to 250° C., preferably from 30 to 160° C., particularly preferably from 90 to 120° C. The CO partial pressure is generally from 1 to 60 bar, preferably from 20 to 55 bar, particularly preferably from 40 to 55 bar. Preferably, the reactor 7 used is a loop reactor.

A portion of the reaction solution is continuously taken off from the reactor 7 and transferred to a separation unit 9. If the separation unit 9 used is a distillation column, the catalyst is taken off overhead and recycled in whole or in part to the reactor 7. Water is taken off from the separation unit 9 via the outlet line 4. The outlet line is either a side stream take off of a dividing wall column or the bottom take off of the upper column if two columns are connected together. The product of value metal formate is produced as melt at the bottom of the column and is taken off from there and transferred to a mixer 8. Via a feedline 5, formic acid is continuously added to the mixer 8 at a concentration of ≧80% by weight, preferably ≧94% by weight, particularly preferably ≧98% by weight. The product of value, the metal formate/formic acid mixture, is taken off from the mixer 8 via an outlet line 6. Whereas in the distillation column 9 generally temperatures from 50 to 220° C., preferably from 60 to 150° C., particularly preferably from 90 to 120° C., and pressures from 0.2 to 15 bar, preferably from 1 to 5 bar, particularly preferably from 1 to 3 bar, prevail, the temperature in the mixer 8 is from 8 to 120° C., preferably from 50 to 120° C., particularly preferably from 90 to 120° C., very particularly preferably from 100 to 120° C., and the pressure is from 0.5 to 10 bar, preferably from 1 to 5 bar, particularly preferably from 1 to 2 bar.

The metal formate/formic acid mixture is then, via a heat exchanger, brought to a storage temperature of from 0 to 40° C., preferably from 20 to 30° C., particularly preferably from 20 to 25° C., and supplied to the store or despatched in solid form (not shown). The heat exchanger used is, for example, a fluidized bed.

If the inventive process is carried out in a plant according to FIG. 2, the individual process steps are carried out in the time sequence a), c) and b). CO or a CO-containing gas mixture is passed into a reactor 7 via an inlet 1 from the bottom and unreacted CO is taken off from the reactor at the top and is recycled in whole or in part via a line 10 back to the reactor 7. An aqueous metal hydroxide solution, to which is added a catalyst stream via a feedline 3, is fed via a feedline 2 to the reactor 7 from the top. Preferably, the reactor 7 used is a loop reactor. The CO partial pressure during the reaction is generally from 1 to 60 bar, preferably from 20 to 55, particularly preferably from 40 to 55 bar. The temperatures in the reactor 7 are generally from 20 to 250° C., preferably from 30 to 160° C., particularly preferably from 90 to 120° C., and the pressures from 1 to 50 bar, preferably from 3 to 40 bar, particularly preferably from 8 to 20 bar. A portion of the reaction solution is continuously taken off from the reactor 7 and transferred to a mixer 8. Via a feedline 5, formic acid is passed into the mixer 8, with the formic acid being at a concentration of ≧80% by weight, preferably 24 94% by weight, particularly preferably ≧98% by weight of aqueous solution.

The temperature during the mixing operation in the mixer 8 is generally from 8 to 120° C., preferably from 50 to 120° C., particularly preferably from 90 to 120° C., very particularly preferably from 100 to 120° C., and the pressure from 0.5 to 10 bar, preferably from 1 to 5 bar, particularly preferably from 1 to 2 bar. The resultant mixture is continuously transferred to a distillation column 9, which consists of formic acid-resistant material. The distillation is generally carried out at temperatures from 50 to 140° C., preferably from 80 to 120° C., particularly preferably from 90 to 120° C. The pressure in the distillation column 9 is generally from 0.2 to 15 bar, preferably from 1 to 5 bar, particularly preferably from 1 to 3 bar. The catalyst is continuously taken off from the distillation column overhead and is preferably recycled in whole or in part to the reactor 7. The water is taken off from the side stream part of the distillation column 4 and either reused in whole or in part for producing aqueous metal hydroxide solutions or is fed in whole or in part to other treatment devices. The metal formate/formic acid mixture wanted is produced at the bottom of the column.

This mixture is then, via a heat exchanger, brought to a storage temperature of from 0 to 40° C., preferably from 20 to 30° C., particularly preferably from 20 to 25° C., and fed to the storage or despatched in solid form. The heat exchanger used is, for example, a fluidized bed.

List of designations

In FIGS. 1 and 2, the designations have the following meanings:

| | |
|---|---|
| 1 | CO inlet; |
| 2 | Feedline for the aqueous metal hydroxide solution; |
| 3 | Feedline for the catalyst; |
| 4 | Outlet line for water; |
| 5 | Feedline for the formic acid; |
| 6 | Outlet line for the product; |
| 7 | Reactor; |
| 8 | Mixer; |
| 9 | Separation unit; |
| 10 | Line. |

We claim:

1. A process for preparing metal formate/formic acid mixtures comprising the following steps:
a) Reacting at least one metal hydroxide with carbon monoxide (CO) or a CO-containing gas in the presence of a catalyst in an aqueous solution to give the corresponding metal formate(s);
b) separating off the water and catalyst by distillation;
c) adding formic acid;
d) if appropriate, adjusting to storage temperature the temperature of the metal formate/formic acid mixture obtained using steps a) to c);
with, in step a), the metal hydroxides being selected from the group consisting of the hydroxides of alkali metals and the hydroxides of alkaline earth metals and step c) also being able to be carried out in time before step b).

2. The process as claimed in claim 1, wherein the metal hydroxides are selected from the group consisting of the hydroxides of sodium, potassium and calcium.

3. The process as claimed in claim 1, wherein the catalyst in step a) is at least one catalyst selected from the group consisting of alcohol and formic esters.

4. The process as claimed in claim 1, wherein the catalyst is at least one catalyst selected from the group consisting of unbranched C₁-C₄ alkanol, branched C₃-C₄ alkanol and formic esters thereof.

5. A process as claimed in claim 1, wherein the catalyst is methanol.

6. The process as claimed in claim 1, wherein the reaction in step a) is carried out in a device selected from the group consisting of a stirred tank having a gas-introduction device, a bubble column, and a loop reactor.

7. The process as claimed in claim 1, wherein the addition in step c) is performed in a stirred tank, extruder or mixer, with or without heat-exchange devices, and the mixing operation is performed using a reaction mixing pump, a mixing nozzle, or a fluidized bed.

8. The process as claimed in claim 1, wherein step a) is carried out at temperatures of from 20 to 250° C. and a pressure of from 1 to 50 bar.

9. The process as claimed in claim 1, wherein step b) is carried out at temperatures of from 50 to 220° C. and a pressure of from 0.2 to 15 bar.

10. The process as claimed in claim 1, wherein step c) is carried out at temperatures of from 8 to 120° C. and a pressure of from 0.5 to 10 bar.

11. The process as claimed in claim 1, wherein the storage temperature in step d) is a temperature from 0 to 40° C.

12. The process as claimed in claim 1, wherein step a) is carried out at temperatures of form 20 to 250° C. and a pressure of from 1 to 50 bar, wherein step b) is carried out at temperatures of from 50 to 220° C. and a pressure of from 0,2 to 15 bar, wherein step c) is carried out at temperatures of from 8 to 120° C. and a pressure of from 0,5 to 10 bar, and wherein the storage temperature in step d) is a temperature from 0 to 40° C.
